(19) United States
(12) Patent Application Publication
(10) Pub. No.: US 2011/0009670 A1
(43) Pub. Date: Jan. 13, 2011

(54) CYCLOHEXANEDIMETHANAMINE BY DIRECT AMINATION OF CYCLOHEXANEDIMETHANOL

(75) Inventors: Terry L. Renken, Conroe, TX (US); Howard P. Klein, Austin, TX (US)

Correspondence Address: HUNTSMAN PETROCHEMICAL LLC, 10003 WOODLOCH FOREST DRIVE, THE WOODLANDS, TX, 77380, US

(73) Assignee: Huntsman Petrochemical LLC, The Woodlands, TX (US)

(21) Appl. No.: 12/919,435

(22) PCT Filed: Mar. 9, 2009
(86) PCT No.: PCT/US09/36458

§ 371 Date: Aug. 25, 2010

Related U.S. Application Data
(60) Provisional application No. 61/035,234, filed on Mar. 10, 2008.

(51) Int. Cl. C07C 209/04

(52) U.S. Cl. 564/447

(57) ABSTRACT

Embodiments of the present invention include methods for producing a cyclohexanedimethanamine by a reductive amination process.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of the Invention

The present invention relates generally to methods of producing amines and more particularly to the direct (one-step) amination of cyclohexanedimethanol to produce cyclohexanedimethanamine (CHDMA).

2. Background of the Invention

Traditional methods of turning alcohols into amines involve multiple step reactions. One method to produce 1,3-CHDMA involves using a nitrile route starting with meta xylene, then hydrogenating that intermediate to meta-xylenediamine (MXDA), which is further hydrogenated to the 1,3-CHDMA. Another method involves producing a mixture of 1,3-CHDMA and 1,4-CHDMA via reduction of a mixture of 1,3- and 1,4-benzenedicarboxylic acids to the two aldehydes, then reductive amination to the mixture of 1,3- and 1,4-CHDMA. These methods require multiple steps that decrease manufacturing efficiency. Also the above methods may only allow certain types of cyclohexanedimethanamine to be produced.

### BRIEF SUMMARY OF SOME OF THE PREFERRED EMBODIMENTS

Embodiments of the present invention disclose a method for producing a cyclohexanedimethanamine by contacting a catalyst with a pressurized amination mixture that includes a cyclohexanedimethanol, an amino compound, and a hydrogen.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other methods for carrying out the same purposes of the present invention. It should also be realized by those skilled in the art that such equivalent methods do not depart from the spirit and scope of the invention as set forth in the appended claims.

### DETAILED DESCRIPTION OF THE PREFERRED EMBODIMENTS

Embodiments of the present invention disclose a method for producing a cyclohexanedimethanamine product by a reductive amination process. The method provides a cyclohexanedimethanol and contacts the cyclohexanedimethanol with an amino compound under a pressure that is greater than atmospheric pressure to form a pressurized amination mixture. The pressurized amination mixture contacts a catalyst.

The present invention includes a cyclohexanedimethanol, an amino compound, and optionally a hydrogen. The cyclohexanedimethanol may include 1,4-cyclohexanedimethanol, 1,3-cyclohexanedimethanol, the cis and trans versions thereof, and combinations thereof. One skilled in the art, with the benefit of this disclosure, will recognize appropriate cyclohexanedimethanols for use in this invention.

The amino compounds useful in this invention include any compounds that can contribute a reactive amine species. In embodiments of the present invention the amino compounds may include ammonia, organic amines, or combinations thereof. One skilled in the art with the benefit of this disclosure will recognize other appropriate amino compounds for use in this invention.

The reaction mixture also includes hydrogen. In one embodiment, hydrogen gas is used. Other compounds that can donate a reactive hydrogen species may be used. One skilled in the art, with the benefit of this disclosure will recognize other appropriate hydrogen sources for use in this invention.

The cyclohexanedimethanol, amino compound, and the hydrogen are contacted under a pressure that is greater than atmospheric pressure to form a pressurized amination mixture. In one embodiment, the pressurized amination mixture has a pressure in the range of about 300 pounds per square inch (psi) to about 5000 psi. In another embodiment, the pressurized amination mixture has a pressure of about 1800 psi to about 2000 psi. One skilled in the art, with the benefit of this disclosure will recognize appropriate pressures to use in embodiments of the present invention.

In embodiments of the present invention, the temperature of the pressurized amination mixture has a temperature in the range of about 150 degrees Celsius (° C.) to about 250° C. In an embodiment, the pressurized amination mixture has a temperature of about 200° C. One skilled in the art, with the benefit of this disclosure will recognize appropriate temperatures for use in the present invention.

In embodiments of the present invention, the pressurized amination mixture described above is contacted with a catalyst. The catalysts useful in this invention include nickel, cobalt, copper, platinum, tin, chromium, zirconium, other metals that are known to catalyze the amination of alcohols, and combinations thereof, whether used separately or in combination with one another. The catalysts may be unsupported or supported on materials such as alumina, silica, silica-alumina, zirconia, titania, carbon, and other known supports. The catalyst may be in tablet foam. In one embodiment of the present invention, the catalyst is a nickel, copper, and chromium catalyst. In another embodiment, the catalyst comprises nickel, copper, tin, and zirconium. One skilled in the art, with the benefit of this disclosure will recognize appropriate catalysts for use in embodiments of the present invention.

The catalysts of the present invention may also include promoters. Promoters generally include materials that enhance catalytic activity of a catalyst. Promoters may include chromium, iron, zinc, zirconium, manganese, molybdenum, and combinations thereof.

The reaction of the pressurized amination mixture and the catalyst forms a cyclohexanedimethanamine product. As more fully described in the Examples below, the reductive amination process may create more than one chemical species. These species may include cyclohexanedimethanamine as well as a reaction effluent mixture of other side products which may include monoamine derivative(s) of cyclohexanedimethanol, unreacted diol(s), light bicyclic amines, and heavies.

In another embodiment of the present invention, the cyclohexanedimethanamine product is distilled. In an embodiment, the distillation includes separating the cyclohexanedimethanamine product from the reaction effluent mixture. This may also include separating the monoamine derivative(s) of cyclohexanedimethanol and unreacted diol(s) from other chemical species that are in present the reaction effluent mixture, such as light bicyclic amines and heavies. One skilled in the art will recognize that distillation and separating methods often do not result in complete purity of chemical species. Therefore distilled chemicals may still be tainted with minute amounts of other chemical species.

In another embodiment of the present invention, the monoamine derivative(s) of cyclohexanedimethanol and unreacted diol(s) are run through the amination process again to form the cyclohexanedimethanamine. This may be accomplished by recycling the recovered monoamine derivative(s) of cyclohexanedimethanol and unreacted diol(s) back into the method for producing a cyclohexanedimethanamine product. This method may include contacting fresh cyclohexanedimethanol with the amino compound and the recovered monoamine derivative(s) of cyclohexanedimethanol, recovered unreacted diol(s) and hydrogen under a pressure that is greater than atmospheric pressure to form a second pressurized amination mixture and contacting the second pressurized amination mixture with the catalyst.

The cyclohexanedimethanamine products produced by methods disclosed hereunder may be used in a variety of applications. For example, the 1,4-CHDMA is an aliphatic diamine useful in several polymer applications including without limitation, epoxy curing, polyamides, and polyureas. The diisocyanate derivative of 1,4-CHDMA is also useful in polyurethane and polyurea applications.

### EXAMPLES

In these Examples, 1,4-Cyclohexanedimethanamine (CHDMA), a mixture of the trans and cis isomers, was readily prepared in high selectivity by a one-step amination of 1,4-cyclohexanedimethanol with ammonia and a nickel-based reductive amination catalyst. This invention offers an attractive alternative to the multiple-step nitrile route in the preparation of 1,4-CHDMA.

### Example 1

Amination of 1,4-Cyclohexanedimethanol. The amination was performed in a 100 cubic centimeter (cc) tubular reactor fully charged with 100 cc of Ni—Cu—Cr catalyst (Ni-2715, ⅛diam×⅛ inch pellets, 74% Ni, 12% Cu, 2% Cr). 1,4-cyclohexanedimethanol (CHDM-D, 99%; Eastman Chemicals Company, Kingsport, Tenn.), ammonia and hydrogen were each continuously fed to the heated reactor. The CHDM-D and ammonia feed rates were kept constant at 90 gallons per hour (g/hr) and 210 g/hr, respectively. The hydrogen was fed at rates of 1.7, 2.8, 5.6 and 11.1 liters/hour (l/hr) to observe any effects on conversion or selectivity. Samples were taken at reactor temperatures of 190° C., 200° C., 210° C. and 220° C. at each hydrogen rate. Reactor pressure was maintained at 2500 pounds per square inch gauge (psig) with a back pressure regulator. Reactor effluent samples were analyzed by gas chromatography and titrated for secondary amine content. Results are shown in Table 1 below. In Table 1: “3-Aza” refers to 3-azabicyclo[3.2.2]nonane; “Monoamine” refers to 1-hydroxymethyl-4-aminomethylcyclohexane; “Diamine” refers to 1,4-cyclohexanedimethanamine (1,4-CHDMA); “Residue” refers to any higher boiling materials than 1,4-CHDMA. Residue was determined by secondary amine content, subtracting the secondary amine contribution from 3-azabicyclo[3.2.2]nonane.

TABLE 1
Amination of 1,4-Cyclohexanedimethanol

| Run A: H₂, @ 2.8 l/hr | | | | |
|---|---|---|---|---|
| Sample 6880-8- | 1A | 2A | 3A | 4A |
| Mid Rx Temp, ° C. | 181 | 190 | 201 | 209 |
| CHDM-D, g/hr | 90.0 | 88.0 | 92.0 | 86.0 |
| Ammonia, g/hr | 213.0 | 212.0 | 210.0 | 212.0 |
| % CHDM-D Conv | 60.9 | 80.5 | 90.1 | 95.1 |
| Wt % Selectivities: | | | | |
| 3-Aza | 0.81 | 1.01 | 1.53 | 1.52 |
| Diamine | 38.18 | 56.30 | 65.63 | 64.70 |
| Monoamine | 60.29 | 40.17 | 24.78 | 15.23 |
| Residue | 0.72 | 2.52 | 8.07 | 18.56 |
| Run B: H₂ @ 5.6 l/hr | | | | |
| Sample 6880-8- | 1B | 2B | 3B | 4B |
| Mid Rx Temp, ° C. | 180 | 190 | 201 | 210 |
| CHDM-D, g/hr | 88.0 | 87.0 | 86.0 | 87.0 |
| Ammonia, g/hr | 210.0 | 208.0 | 202.0 | 210.0 |
| % CHDM-D Conv | 47.2 | 73.8 | 90.0 | 96.0 |
| Wt % Selectivities: | | | | |
| 3-Aza | 0.61 | 0.94 | 1.70 | 2.55 |
| Diamine | 28.51 | 51.24 | 69.55 | 72.80 |
| Monoamine | 69.93 | 47.17 | 24.03 | 12.21 |
| Residue | 0.95 | 0.64 | 4.72 | 12.44 |
| Run C: H₂, @ 1.7 l/hr | | | | |
| Sample 6880-8- | 1C | 2C | 3C | 4C |
| Mid Rx Temp, ° C. | 181 | 190 | 201 | 211 |
| CHDM-D, g/hr | 92.0 | 87.0 | 92.0 | 96.0 |
| Amnaonia, g/hr | 204.0 | 209.0 | 211.0 | 208.0 |
| % CHDM-D Conv | 45.6 | 62.8 | 76.2 | 91.7 |
| Wt % Selectivities: | | | | |
| 3-Aza | 0.32 | 0.61 | 1.14 | 2.19 |
| Diamine | 25.27 | 38.00 | 50.09 | 60.29 |
| Monoamine | 73.84 | 60.44 | 45.69 | 21.06 |
| Residue | 0.57 | 0.95 | 3.07 | 16.46 |
| Run D: H₂, @ 11.1 l/hr | | | | |
| Sample 6880-8- | 1D | 2D | 3D | 4D |
| Mid Rx Setpt, C | 180 | 191 | 201 | 210 |
| CHDM-D, g/hr | 88.0 | 88.0 | 86.0 | 86.0 |
| Ammonia, g/hr | 210.0 | 208.0 | 213.0 | 208.0 |
| % CHDM-D Cony | 44.0 | 70.6 | 87.4 | 95.9 |
| Wt % Selectivities: | | | | |
| 3-Aza | 0.65 | 1.05 | 1.82 | 2.66 |
| Diamine | 26.09 | 48.81 | 66.40 | 69.46 |
| Monoamine | 72.37 | 47.67 | 25.45 | 11.67 |
| Residue | 0.89 | 2.47 | 6.34 | 16.21 |

### Example 2

Isolation of 1,4-Cyclohexanedimethanamine by distillation of amination effluent. Reactor effluent was collected using the same reactor described in Example 1 and the following conditions: 90 g/hr CHDM-D, 210 g/hr ammonia, 5.6 l/hr hydrogen, 2500 psig, 200° C. Capillary GC analysis of the effluent was used to determine the following results as shown in Table 2.

TABLE 2
Reactor Effluent Results

| | | |
|---|---|---|
| | % CHDM-D conversion | 89.6 |
| % Selectivities: | Cyclohexylmethylamine | 0.11 |
| | Cyclohexylmethanol | 0.07 |
| | 3-Azabicyclo[3.2.2] nonane | 1.54 |
| | Diamine | 69.00 |
| | Monoamine | 26.78 |
| | Residue | 2.48 |

A sample of the reactor effluent was fractionally distilled to afford pure diamine (bp 135° C., 25 mm) and monoamine (167° C., 25 mm).

While it has not yet been demonstrated, it is believed that the mixtures of CHDM-D diol and the monoamine intermediate can be fed to the amination reactor to produce diamine product. Thus, monoamine may be isolated and taken as a product or recycled back to the amination reactor to make diamine.

Although the present invention and its advantages have been described in detail, it should be understood that various changes, substitutions and alterations may be made herein without departing from the spirit and scope of the invention as defined by the appended claims.

What is claimed is:

1. A method for producing a cyclohexanedimethanamine product by a reductive amination process comprising the steps of:
a. providing a cyclohexanedimethanol, an amino compound, and a hydrogen;
b. contacting the cyclohexanedimethanol with the amino compound and the hydrogen under a pressure that is greater than atmospheric pressure to form a pressurized amination mixture; and
c. contacting the pressurized amination mixture with a catalyst.

2. The method of claim 1 wherein the amino compound is selected from the group consisting of: ammonia, an organic amine, and combinations thereof.

3. The method of claim 1 wherein the catalyst comprises metals selected from the group consisting of: nickel, cobalt, copper, platinum, tin, zirconium, and combinations thereof.

4. The method of claim 1 wherein the catalyst comprises nickel, copper, and chromium.

5. The method of claim 1 wherein the catalyst comprises nickel, copper, tin and zirconium.

6. The method of claim 1 wherein the catalyst further comprises a promoter.

7. The method of claim 6 wherein the promoter comprises chromium, iron, zinc, zirconium, manganese, molybdenum, and combinations thereof.

8. The method of claim 1 wherein the cyclohexanedimethanol is selected from the group consisting of: 1,4-cyclohexanedimethanol, 1,3-cyclohexanedimethanol, and combinations thereof.

9. The method of claim 1 wherein the pressure that is greater than atmospheric pressure comprises a pressure in the range of about 300 psi to about 5000 psi.

10. The method of claim 1 wherein the pressure that is greater than atmospheric pressure comprises a pressure in the range of about 1800 psi to about 2000 psi.

11. The method of claim 1 wherein the pressurized amination mixture has a temperature in the range of about 150° C. to about 250° C.

12. The method of claim 1 wherein the pressurized amination mixture has a temperature of about 200° C.

13. The method of claim 1, further comprising the step of distilling the cyclohexanedimethanamine product.

14. The method of claim 13, wherein distilling the cyclohexanedimethanamine product comprises separating a reaction effluent mixture from the cyclohexanedimethanamine product.

15. The method of claim 14, wherein separating the reaction effluent mixture further comprises separating a monoamine derivative of cyclohexanedimethanol and an unreacted diol from the reaction effluent mixture.

16. The method of claim 15, further comprising the steps of:
a. contacting the cyclohexanedimethanol with the amino compound, the monoamine derivative of cyclohexanedimethanol, the unreacted diol and the hydrogen under a pressure that is greater than atmospheric pressure to form a second pressurized amination mixture; and
b. contacting the second pressurized amination mixture with the catalyst.
